(19) United States
(12) Patent Application Publication
(10) Pub. No.: US 2016/0346472 A1
(43) Pub. Date: Dec. 1, 2016

(54) In-Line Fluid Injection System Comprising a Manifold and Contrast Source Valve for Controlled Injection of Multiple Fluids

(71) Applicant: AngioDynamics, Inc., Latham, NY (US)

(72) Inventors: James J. Mitchell, Ballston Spa, NY (US); Brett Zubiate, Duxbury, MA (US); Timothy Hall, Wilton, NY (US); Reginald Harold Day, Lynn Haven, FL (US)

(73) Assignee: AngioDynamics, Inc., Latham, NY (US)

(21) Appl. No.: 15/168,878

(22) Filed: May 31, 2016

Related U.S. Application Data
(60) Provisional application No. 62/167,932, filed on May 29, 2015.

(51) Int. Cl. A61M 5/19; A61M 5/00; A61M 39/10; A61M 39/22; A61M 39/24

(52) U.S. Cl. CPC A61M 5/19; A61M 39/223; A61M 39/24; A61M 39/1011; A61M 5/007; A61M 2039/229; A61M 2039/242; A61M 2039/2493; A61M 2202/0085; A61M 2005/1787

(57) ABSTRACT

The present disclosure relates to systems and methods for injecting multiple fluids into the vasculature of a patient using a minimal number of valve and stopcock manipulations. In particular, the present disclosure relates to a fluid injection system that combines a contrast source valve and a manifold in-line to a manual or automated syringe assembly for alternately injecting contrast and saline solutions into the vasculature of a patient.

### CROSS-REFERENCE TO RELATED APPLICATIONS

This application claims priority to U.S. Provisional Application No. 62/167,932 filed on May 29, 2015 and is incorporated herein by reference.

### FIELD

The present disclosure relates generally to systems and methods for injecting multiple fluids into the vasculature of a patient with a minimal number of valve and stopcock manipulations. More specifically, the present disclosure relates to an injection system comprising a catheter, manifold and dual-check valve connected in-line to a manual or automated syringe assembly for alternately injecting contrast and saline solutions into the vasculature of a patient.

### BACKGROUND

Angiography is a procedure in which radiographic images are used to detect abnormalities or restrictions within the vasculature. These radiographic images are obtained by injecting radiographic contrast solution (i.e., contrast) through a catheter into a vein or artery of the patient prior to taking an X-ray of the portion of the vasculature being examined. The X-rays are absorbed by the contrast to produce a radiographic outline or image of the target region.

The amount and/or rate of contrast administered to the patient is typically controlled by a manual or automatic injection system connected to the proximal end of the catheter. To monitor the pressure within the vessel or artery being examined, the catheter is in fluid communication with a pressure transducer during the angiography procedure. The pressure transducer and contrast injection system are typically connected to the catheter through separate lines that pass through a manifold that allows the physician to isolate/protect the delicate pressure transducer from elevated pressures associated with injecting the viscous contrast solution. Specifically, many pressure transducers can be damaged if subjected to pressure in excess of 75 psi. As even a hand-operated syringe is capable of generating pressures in excess of 200 psi, isolation of the pressure transducer is essential to avoid transducer failure and loss of patient monitoring capability.

One solution to this problem is to use a manifold that does not allow contrast to be injected while the pressure transducer is in communication with the catheter. For example, the manifold may include a rotatable stopcock that allows the catheter to be in fluid communication with either the pressure transducer or the contrast injection source, but not both. To monitor the patient's vascular pressure following each injection of contrast, the physician must manually rotate the stopcock back-and-forth between the two positions throughout the angiography procedure. Since a single angiography procedure may require more than 200 separate contrast injection steps, it is not uncommon for the physician to forget to return the stopcock to the vascular pressure monitoring position. As a result, the ability to monitor the pressure within the artery or vessel may be interrupted for unnecessarily long periods of time. In addition to potential negative consequences for the patient, such disruptions require other members of the medical team to interrupt the physician and tell him or her to turn the stopcock back again, which may cause an unnecessary distraction to the physician during a delicate medical procedure.

This problem is further complicated by the fact that angiography procedures often include intermittent injections of saline solution for a variety of purposes. For example, since a reliable angiographic image may be obtained using contrast diluted as much as 50-75%, physicians commonly administer saline to reduce the total amount of expensive contrast agent required for each procedure. Diluting the concentration of contrast with saline is also safer for certain patients, such as those with renal insufficiency, because it reduces the burden on the kidneys to filter contrast from the blood. Saline may also be administered to flush the catheter line of contrast or other medications, to prime the manifold, to dilute the concentration of a medication within the patient and/or as a slow injection to maintain a vessel in an open configuration. It is important to flush the catheter line with saline since the high viscosity of contrast tends to dampen the patients' pressure waveforms, resulting in unreliable measurements by the pressure transducer. Saline provides a more desirable mechanical response to pressure waveforms and allows more accurate transmission of the patient's intravascular pressure to the pressure transducer. As with the injection of contrast, the pressure transducer must be isolated prior to each injection of saline and then returned to vascular pressure monitoring position. Naturally, these additional steps increase the number of stopcock manipulations required by the physician and further increase the likelihood of error.

In addition to the potential for human error, the numerous valve and stopcock manipulations required for an angiography procedure represent a significant physical burden. The repetitive strain exerted on the user's hands and/or fingers during these long procedures can result in temporary discomfort as well as long term problems such as carpel tunnel syndrome. This is especially true of conventional injection systems that tend to include small knobs that are difficult to grasp and turn each time a manifold is switched between an open and closed position. These problems are even more pronounced with manual injection systems that also require the user to retract and depress the plunger of a syringe as the viscous contrast agent is drawn from the contrast source and injected into the patient. For users that perform multiple angiography procedures per day, these repetitive physical demands can easily become problematic. Moreover, since the cost per minute of an operating room suite can average almost $150 per minute, not including anesthesiology costs, the additional time spent performing numerous operations represents a significant financial burden to the healthcare system.

There is a continued need for improved manual and/or automatic fluid injection systems that permit contrast and saline solutions to be alternately injected into the patient's vasculature with a minimal number of valve and stopcock manipulations, while simultaneously allowing the catheter to remain in fluid communication with the pressure transducer.

### SUMMARY

In one aspect, the present disclosure relates to a manifold, comprising: a housing that defines intersecting first, second, third and fourth ports, wherein the non-intersecting end of each port is configured to be fluidly connected to an element that includes a fluid channel; a rotatable stopcock comprising three fluidly connected conduits rotatably disposed at the intersection of the first, second, third and fourth ports; and an actuator attached to the rotatable stopcock. The non-intersecting end of the first port may be fluidly connected to a pressurized saline source. The non-intersecting end of the second port may be fluidly connected to a dual-check valve. The non-intersecting end of the third port may be fluidly connected to a pressure transducer. The non-intersecting end of the fourth port may be fluidly connected to a catheter. The rotatable stopcock may be configured to rotate about four stopcock positions within the manifold. The rotatable stopcock may be rotated by turning the actuator handle. Each of the four stopcock positions preferably blocks fluid communication with a different one of the first, second, third and fourth ports. For example, a first stopcock position may place the second, third and fourth ports in fluid communication while blocking fluid communication with the first port; a second stopcock position may place the first, third and fourth ports in fluid communication while blocking fluid communication with the second port; a third stopcock position may place the first, second and fourth ports in fluid communication while blocking fluid communication with the third port; and a fourth stopcock position may place the first, second and third ports in fluid communication while blocking fluid communication with the fourth port.

In another aspect, the present disclosure relates to a fluid injection system, comprising: a syringe assembly; a contrast source valve fluidly connected to the syringe assembly; a manifold fluidly connected to the contrast source valve; and a catheter fluidly connected to the manifold. The contrast source valve may include a dual-check valve. The contrast source valve may be fluidly connected a contrast source. The manifold may include: a housing that defines intersecting first, second, third and fourth ports, wherein the non-intersecting end of each port is fluidly connected to an element that includes a fluid channel; a rotatable stopcock comprising three fluidly connected conduits rotatably disposed at the intersection of the first, second, third and fourth ports; and an actuator attached to the rotatable stopcock. The non-intersecting end of the first port may be fluidly connected to a pressurized saline source. The non-intersection end of the second port may be fluidly connected to the syringe assembly. The non-intersecting end of the third port may be fluidly connected to a pressure transducer. The non-intersecting end of the fourth port may be fluidly connected to the catheter. A utility port may be fluidly connected between the contrast source valve and the manifold.

### BRIEF DESCRIPTION OF THE DRAWINGS

Non-limiting embodiments of the present disclosure will be described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 shows a schematic view of a fluid injection system, in accordance with an embodiment of the present disclosure.

FIG. 2 shows a magnified view of the dual-check valve of FIG. 1A.

FIGS. 3A-B show the flow of contrast through the contrast source valve and syringe of FIG. 1 as the plunger is retracted (3A) and/or advanced (3B).

FIGS. 4A-B show side (4A) and cut-away (4B) views of the manifold of FIG. 1 in the contrast injection setting.

FIGS. 5A-B show side (5A) and cut-away (5B) views of the manifold of FIG. 1 in the saline flush setting.

FIGS. 6A-B show side (6A) and cut-away (6B) views of the manifold of FIG. 1 in the pressure transducer isolation setting.

FIGS. 7A-B show side (7A) and cut-away (7B) views of the manifold of FIG. 1 in the pressure protection valve flush setting.

FIG. 8 shows a schematic view of the fluid injection system of FIG. 1 that further includes a utility port between the contrast source valve and manifold.

FIG. 9 shows a schematic view of the fluid injection system that includes an integral pressure transducer, in accordance with an embodiment of the present disclosure.

### DETAILED DESCRIPTION OF THE PREFERRED EMBODIMENTS

Before the present disclosure is described in further detail, it is to be understood that the disclosure is not limited to the particular embodiments described, as such may vary. It is also to be understood that the terminology used herein is for the purpose of describing particular embodiments only, and is not intended to be limiting beyond the scope of the appended claims. Unless defined otherwise, all technical terms used herein have the same meaning as commonly understood by one or ordinary skill in the art to which the disclosure belongs. Finally, although embodiments of the present disclosure are described with specific reference systems and methods that utilize manual syringe assemblies for alternately injecting contrast and saline solutions into the vasculature of a patient, it should be appreciated that the present invention is equally pertinent for automated syringe assemblies, and the delivery of solutions other than contrast and saline. As used herein, the term “distal” refers to the end farthest away from the physician or other medical professional when introducing a device into a patient, while the term “proximal” refers to the end closest to the physician when introducing a device into a patient.

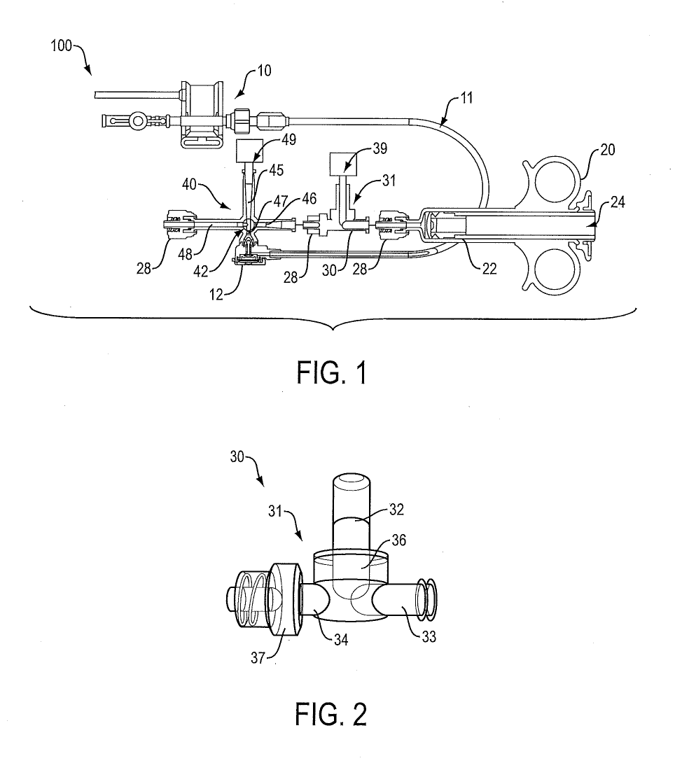

FIG. 1 provides a schematic view of a fluid injection system 100 comprised of various components or subassemblies that may be combined to form the overall system. The fluid injection system allows the physician to monitor vascular blood pressure and perform injections of contrast and saline with a minimal number of valve and stopcock manipulations. The fluid injection system may include, but is not limited to, a pressure transducer 10, a pressure transducer line 11, a pressure protection valve 12, a syringe 20, a contrast source valve 30, a contrast source 39, a manifold 40 that includes a rotatable stopcock valve 42, and a pressurized saline source 49. Any of the components described herein may be fluidly attached by connectors 28 specifically designed for injection systems. For example, standard Luer lock type connectors allow the physician to quickly and reliably connect the components described above into a single assembly by twisting or rotating corresponding male and female members at each connection point. Alternatively, the connectors 28 may be a snap fit or quick connect type connection in which the corresponding ends are fluidly connected via an interference fit.

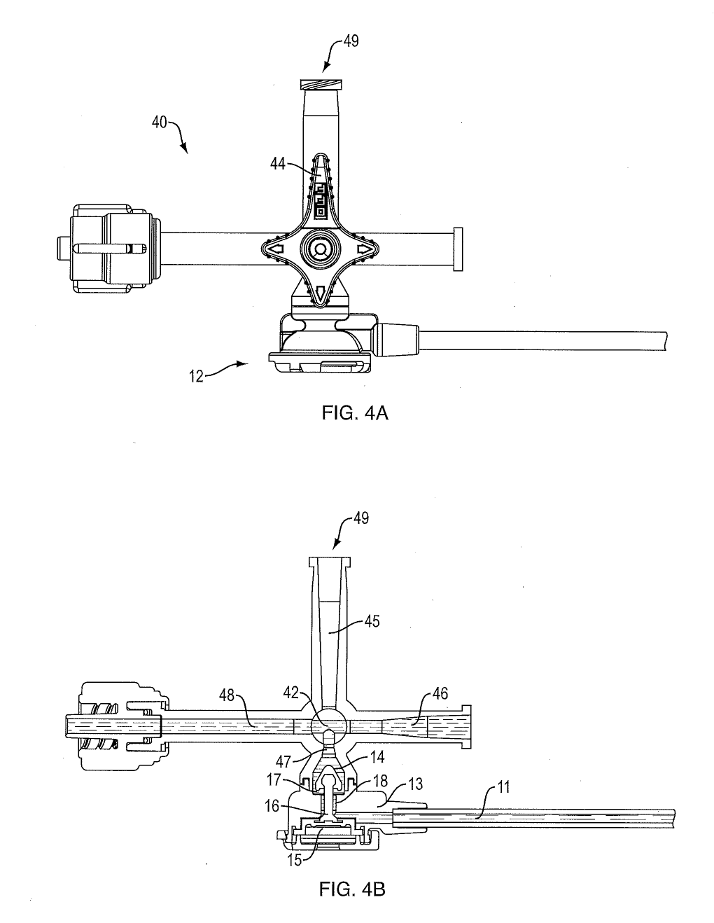

In one embodiment, the rotatable stopcock valve 42 is connected to an external valve actuator 44 (i.e., “handle” or “knob”) that includes an over-sized ergonomic design that facilitates smooth rotation of the stopcock valve 42 under elevated fluid pressures and one-handed operation. As best illustrated in FIGS. 4A-7A, the over-sized valve actuator 44 includes a contoured non-slip surface that allows the physician grasp the valve actuator with one hand, for example, between the thumb and forefinger, for one-handed operation. One advantage of the manifold 40 is that the stopcock 42 is able to rotate under a variety of fluid pressures, including the low fluid pressures associated with manual injection systems and high fluid pressures associated with automated injection systems. This allows the manifold to be used interchangeably with a variety of fluid injection systems, including automated injector systems, manual injector systems, or syringe assemblies from different manufacturers, regardless if the fluid pressure created by the system is of high pressure.

The syringe 20 may be made from various materials able to withstand high temperatures or pressures, including, but not limited to, clear polycarbonate, clear acrylonitrile-butadiene-styrene (abs) or ULTEM®. In one embodiment, the syringe may resemble those generally known in the art. For example, the syringe may include a barrel 22 and plunger 24 that includes multiple ergonomic finger holes 26 into which the physician can place their fingers or thumbs to advantageously achieve comfortable leverage. The syringe 20 may be operated by a manual or automated system in which the plunger 24 is retracted to draw fluid (i.e., contrast) into the barrel 22 of the syringe 20, and then advanced to expel the fluid from the barrel 22 of the syringe 20.

A contrast source valve 30 is positioned between the distal end of the syringe 20 and the proximal end of the manifold 40. One example of the contrast source valve 30 includes a dual-check valve as described throughout the literature, including U.S. Pat. No. 5,104,387, herein incorporated by reference in its entirety. As best shown in FIG. 2, the dual-check valve includes a housing 31 that defines intersecting first 32, second 33 and third 34 fluid channels. The non-intersecting end of each fluid channel is fluidly connected to a separate component of the fluid injection system. For example, the first fluid channel 32 may be fluidly connected to a contrast source 39, the second fluid channel 33 may be fluidly connected to the barrel 22 of syringe 20 and the third fluid channel 34 may be fluidly connected to the manifold 40. A first valve 36 is positioned within the first fluid channel 32 just above the intersection point of the fluid channels. A second valve 37 is positioned within the third fluid channel 34 just below (i.e., downstream of) the intersection point of the fluid channels.

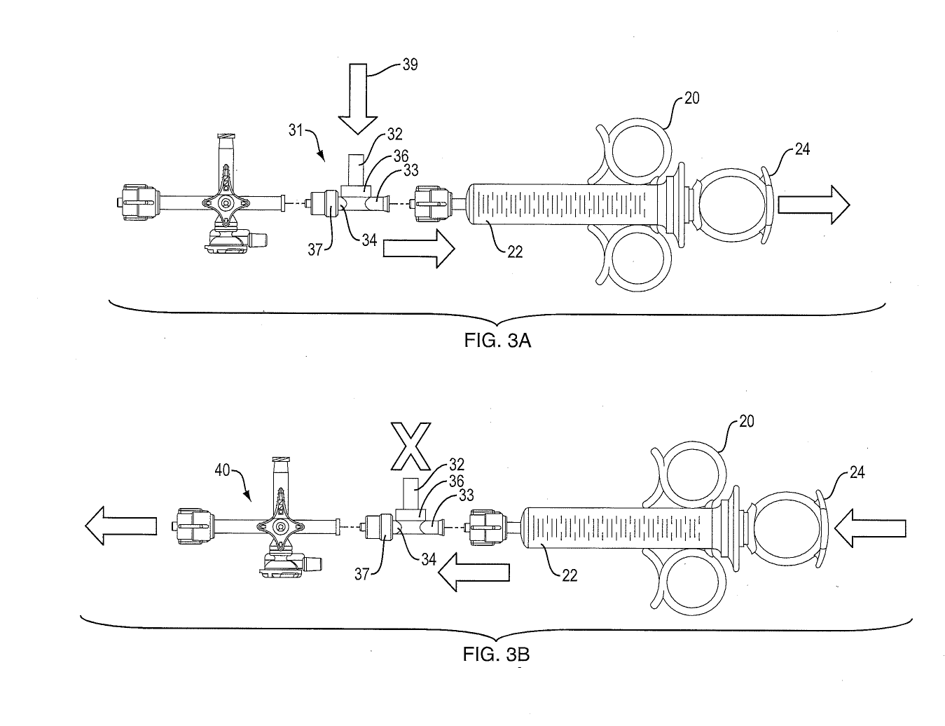

As shown in FIG. 3A, retracting the plunger 24 of the syringe 20 creates negative pressure within the housing 31 that simultaneously urges the first valve 36 to selectively open and the second valve 37 to selectively close. This valve configuration allows contrast to flow from the contrast source 39, through the first 32 and second 33 fluid channels into the barrel 22 of the syringe 20, without entering the third fluid channel 34. As shown in FIG. 3B, depressing the plunger 24 of the syringe 20 simultaneously urges first valve 36 to selectively close and the second valve 37 to selectively open. This valve configuration allows contrast to flow from the barrel 22 of the syringe 20 through the second 33 and third 34 fluid channels to the manifold 40, without entering the first fluid channel 32.

In another embodiment (not shown), the dual-check valve includes a single valve positioned within a main chamber located at the intersection of the first, second and third fluid channels. Retracting the plunger 24 of the syringe 20 creates negative pressure within the main chamber that urges the valve head to selectively open the first fluid channel and simultaneously close the third fluid channel. Thus, contrast flows from contrast source, through the first and second fluid channels into the barrel 22 of the syringe 20 without entering the third fluid channel. Depressing the plunger 24 of the syringe 20 urges the valve to selectively close the first fluid channel and simultaneously open the third fluid channel. Thus, contrast flows from the barrel 22 of the syringe 20, through the second and third fluid channels without entering the first fluid channel.

In one embodiment, the contrast source valve may include a low pressure dual-check valve configured for manual injection systems that generate fluid pressures approaching 200 psi. For example, a low pressure dual-check valve may be connected to a syringe that includes a plunger configured to be manually advanced and retracted using only the leverage generated by the physicians hand(s). In another embodiment, the contrast source valve may include a high pressure dual-check valve configured for automated injection systems capable of generating fluid pressures in excess of 1200 psi. In yet another embodiment, the contrast source valve may include a high pressure automated stopcock valve including, by way of non-limiting example, the automated valve described in U.S. Patent Application Publication No. 20140208251, herein incorporated by reference in its entirety. The high pressure automated contrast source valves may be controlled by a graphical user interface (GUI) such that the physician can control the movement of the syringe plunger by simply touching graphical icons on a display screen.

One advantage of using a dual-check valve to provide a fluid connection between the syringe 20, contrast source 39 and manifold 40 is that the physician is not required to perform any valve manipulations in order to allow the selective flow of contrast into and out of the syringe 20. Instead, the physician simply retracts the plunger 24 to draw contrast from the contrast source 39 into the syringe 20 through the first valve 36, and then compresses the plunger 24 to force the contrast from the syringe 20 towards the manifold 40 through the second valve 37. If the angiography procedures requires additional contrast to be administered to the patient once the syringe has emptied, the physician simply repeats the steps of retracting and depressing the plunger 24 as many times as necessary. Again, regardless of the number of times this process is repeated, the physician does not have to perform a single valve manipulation at the dual-check valve.

Referring again to FIG. 1, a manifold 40 is positioned between the distal end of the contrast source valve 30 and the proximal end of the catheter (not shown). The manifold 40 includes a housing 41 that defines intersecting first 45, second 46, third 47 and fourth 48 ports. The non-intersecting end of each port is fluidly connected to a separate component of the fluid injection system. For example, in one embodiment the first port 45 may be fluidly connected to the pressurized saline source 49; the second port 46 may be fluidly connected to the contrast source valve 30; the third port 47 may be fluidly connected to a pressure protection valve 12 and the fourth port 48 may be fluidly connected to a catheter (not shown).

A four-position rotatable stopcock 42 is located within the manifold 40 at the intersection of the first 45, second 46, third 47 and fourth 48 ports. The rotatable stopcock 42 includes three fluidly connected conduits configured in a T-shape. This configuration allows each of the four rotatable stopcock 42 positions to prevent/block fluid communication with a different one of the intersecting first 45, second 46, third 47 or fourth 48 ports, while simultaneously providing/establishing fluid communication with the remaining three ports. The external valve actuator 44 (i.e., toggle switch) attached to the rotatable stopcock 42 allows the physician to switch the orientation of the stopcock between four distinct fluid injection settings.

As shown in FIGS. 4A-B, the contrast injection setting may be selected by switching the valve actuator 44 to a position that places the rotatable stopcock 42 in fluid communication with the second 46, third 47 and fourth 48 ports. Blocking fluid communication with the first port 45 ensures that contrast flows directly from the contrast source valve 30 through the manifold 40 and catheter (not shown) and into the patient's vasculature.

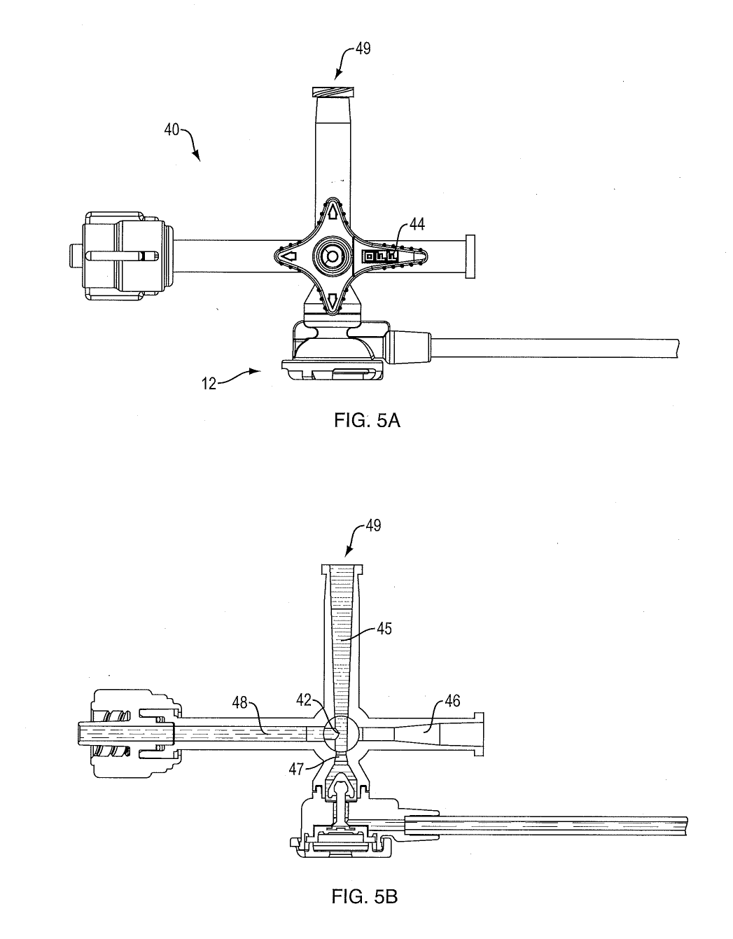

As shown in FIGS. 5A-B, the saline flush setting may be selected by switching the valve actuator to a position that places the rotatable stopcock 42 in fluid communication with the first 45, third 47 and fourth 48 ports. Blocking fluid communication with the second port 46 ensures that saline flows directly from the pressurized saline source 49, through the manifold 40 and catheter (not shown) and into the patient's vasculature without rotating the valve that protects the pressure transducer between the open (“transducer open”) and closed (“transducer closed”) positions.

Importantly, in both the contrast injection and saline flush settings, the rotatable stopcock 42 allows the pressure transducer 10 to remain in fluid communication with the vasculature. This eliminates the need to repeatedly close/open the valve that protects the pressure transducer before/after each contrast injection. As described in U.S. Pat. Nos. 6,896,002 and 6,986,742, both of which are herein incorporated by reference in their entirety, the pressure transducer 10, specifically the flexible diaphragm of the transducer, is protected from potentially damaging high fluid pressures by an integral pressure protection valve 12. Briefly, the pressure protection valve 12 allows two-way fluid communication between the manifold 40 and the pressure transducer 10. The pressure protection valve 12 includes a cap 14, a flexible diaphragm 15, a stem 16, a sealing surface 17, a housing 13, a fluid channel 18 and a pressure transducer line 11. The pressure protection valve 12 is activated when a pressure fluctuation exists between the fluid channel 18 and the pressure transducer line 11 such that the compliant flexible diaphragm 15 deflects away from its original position towards the fluid channel 18. The top of the integrated stem 16 is connected to the diaphragm 15 and the bottom of the stem 16 is connected to the housing 13. Deflection of the diaphragm 15 moves the integrated stem 16 away from the fluid channel 18, causing the cap 14 to engage the sealing surface 17 and establish pressure isolation between the pressurized third port 47 and the pressure transducer 10. The pressure protection valve 12 creates a seal that protects the delicate pressure transducer 10 before fluid pressure within the fluid channel 18 is sufficient to cause damage. Once the pressure of fluid flow through the fluid channel 18 is lowered, the diaphragm 15 returns to the open position, thereby separating the cap 14 from the sealing surface 17 and re-establishing fluid communication between the pressure transducer 10 and the manifold 40.

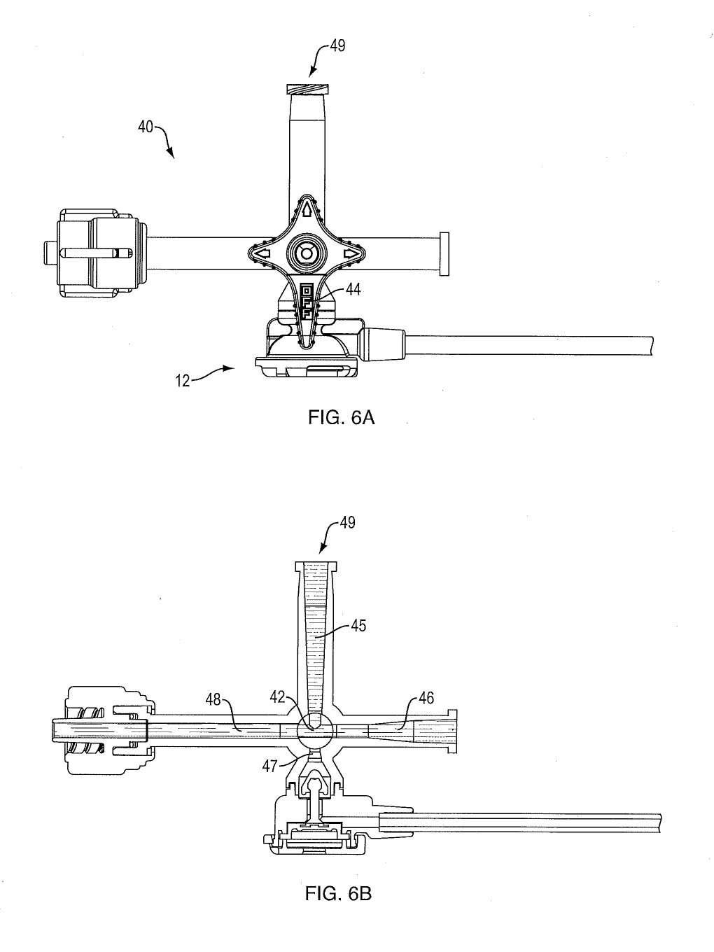

As shown in FIGS. 6A-B, the pressure transducer isolation setting may be selected by switching the valve actuator to a position that places the rotatable stopcock 42 in fluid communication with first 45, second 46 and fourth 48 ports. Intermittent flushing of the catheter line with saline is important because it clears the catheter of particulates and/or thrombosis formation. As stated above, in order to flush the catheter, saline flows directly from the pressurized saline source 49, through the manifold 40 and into the catheter (not shown). The method of pressurizing the saline source is dependent on whether the system being used is a manual system or an automated system. Blocking fluid communication with the third port 47 also allows a damaged pressure transducer 10 to be changed during fluid delivery, as well as allow for the changing of the pressure transducer 10 without having to re-prime the system.

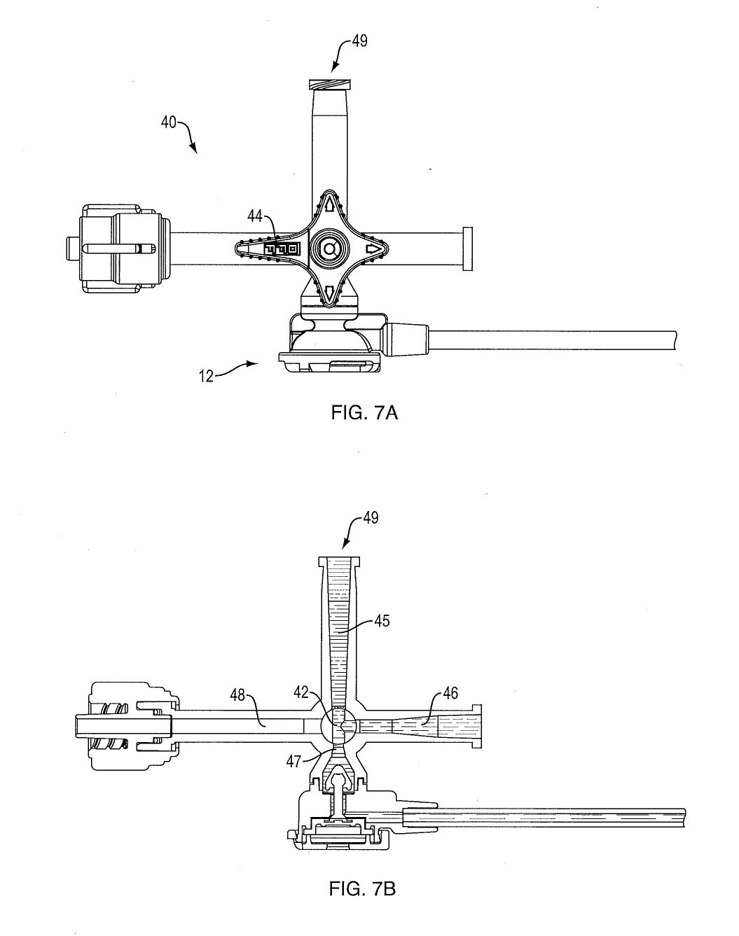

As shown in FIGS. 7A-B, the pressure protection valve flush setting may be selected by switching the valve actuator to a position that places the rotatable stopcock 42 in fluid communication with the first 45, second 46 and third 47 ports. Blocking fluid communication with the fourth port 48 allows the pressure protection valve 12 and pressure transducer line 11 to be flushed with saline to remove any debris or air bubbles that might prevent the pressure transducer from accurately monitoring the patient's vascular pressure.

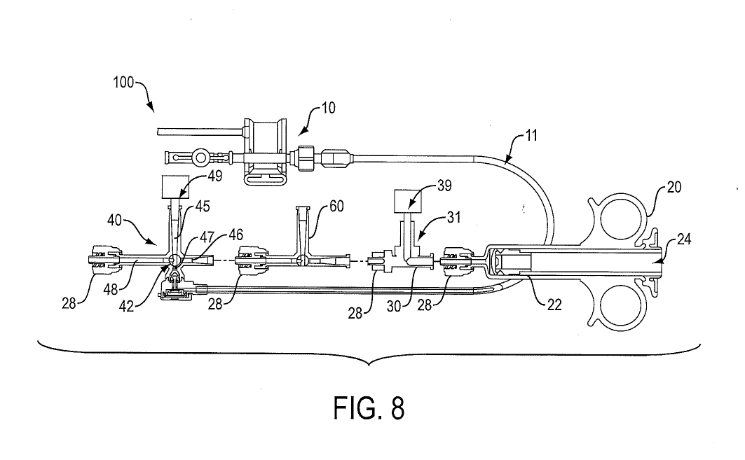

The fluid injection system may further include one or more utility ports that may be used to administer a variety of additional fluids to the patient, draw blood samples from the patient, remove waste and/or purge the delivery system of air bubbles. As illustrated in FIG. 8, in one embodiment a utility port 60 is located between the contrast source valve 30 and manifold 40, the dual-check valve of the contrast source valve 30 preventing fluid, waste or air from traveling into the contrast source 39. It will be appreciated, however, that utility port(s) may be located at various positions within the fluid injection system, including, for example, between the manifold 40 and the catheter (not shown).

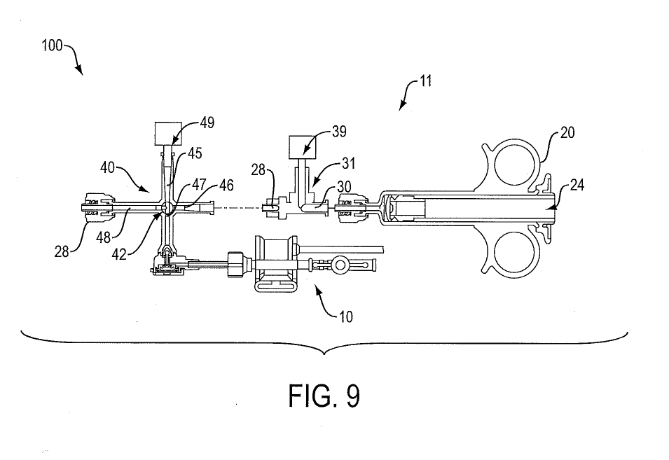

Referring to FIG. 9, in one embodiment, the pressure transducer 10 is connected directly to the pressure protection valve 12 of the manifold 40. Eliminating or significantly shortening the pressure transducer line 11 allows the pressure transducer to be closer to the syringe assembly, rather than 48-60 inches away. Including the pressure transducer as an integral component of the manifold allows the syringe assembly to have a smaller (i.e., more compact) working profile that takes up less space within the operating room. The compact design also requires less packaging during shipping, thereby minimizing waste and recycling costs.

A major advantage of the fluid injection system described herein is the reduced number of valve or stopcock manipulations required at each of the three stages of a typical angiography procedure. As compared to a conventional fluid injection system in which the manifold does not include an integral pressure protection valve that automatically isolates the pressure transducer, and the contrast source is not connected to the syringe via a dual-check valve, the presently disclosed fluid injection systems requires a minimum of five fewer valve and stopcock manipulations per angiography procedure. As described in detail below, the number of stopcock manipulations increases with each successive contrast injection and/or saline flush step.

TABLE 1

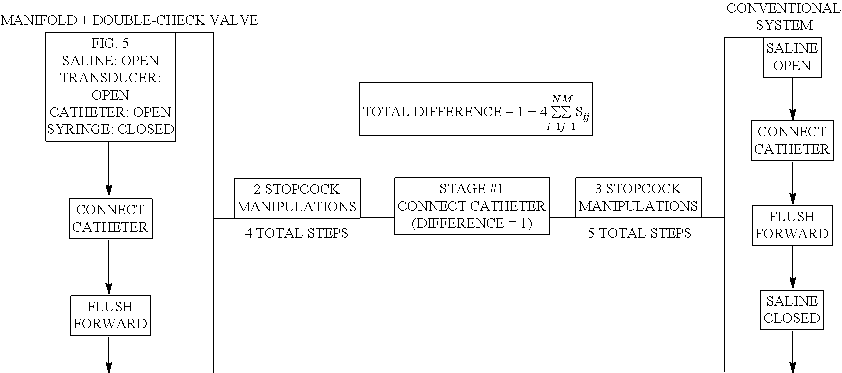

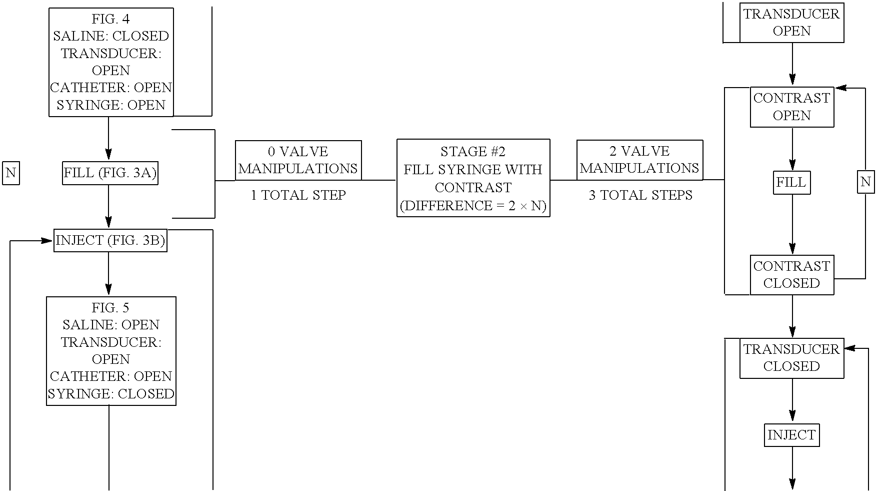

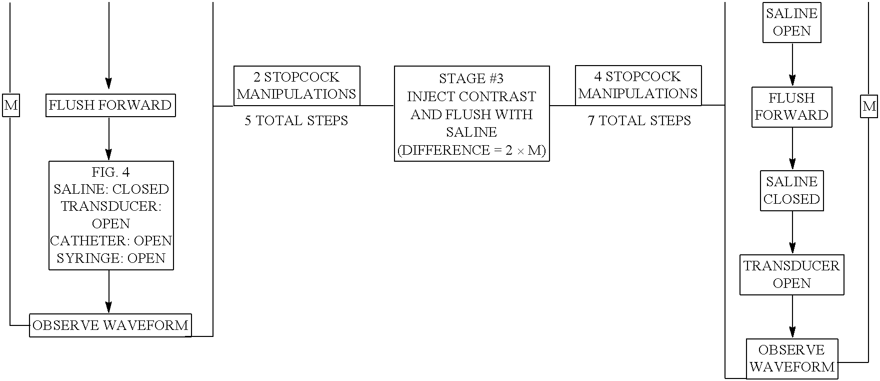

Table 1 provides a side-by-side comparison of the valve and stopcock manipulations required to use the presently disclosed fluid injection system versus a conventional system. For purposes of clarity, the angiography procedure is broadly categorized in three basic stages: connecting the catheter to the manifold (Stage #1), filling the syringe with contrast (Stage #2) and injecting the contrast from the syringe into the patient, followed by a saline flush of the manifold (Stage #3). Throughout the procedure, each valve or stopcock manipulation that requires the physician to manually rotate a stopcock or open/close a valve is indicated by a shaded box.

### Stage #1

The first set of brackets outlines the stopcock manipulations required to connect the catheter to a wet manifold. Referring to the presently disclosed fluid injection system, the first manipulation is to rotate the stopcock to place the manifold in the saline flush setting (FIGS. 5A-B), which blocks fluid communication with the syringe while maintaining fluid communication with the pressure transducer. The catheter is then connected to the manifold (“connect catheter”), and the manifold and catheter are flushed with saline (“flush forward”). Importantly, the pressure transducer is protected from elevated pressures during the saline flush by the pressure protection valve that is integral to the manifold. The stopcock is then rotated 90° counter-clockwise to place the manifold in the contrast injection setting (FIGS. 4A-B), which simultaneously opens fluid communication with the syringe and blocks fluid communication with the saline source while maintaining fluid communication with the pressure transducer. Thus, a total of two stopcock manipulations are required to connect a catheter to the presently disclosed manifold.

By comparison, the first stopcock manipulation required of the conventional fluid injection system is to rotate the manifold to a position that establishes fluid communication with the saline source (“saline open”). The catheter is then connected to the manifold (“connect catheter”), and manifold and catheter are flushed with saline (“flush forward”). Importantly, because the pressure transducer is separate from the manifold, and therefore not protected by an integral pressure protection valve, it must be isolated during the saline flush (“flush forward”) by a separate valve maintained in a closed position. Following the saline flush, the manifold is rotated such that fluid communication with the saline source is blocked (“saline closed”). An additional stopcock manipulation is then required to open the valve that protects the pressure transducer (“transducer open”) so that the patient's vascular pressure can be monitored. Thus, a total of three stopcock manipulations are required to connect a catheter to the conventional manifold. The ability of the presently disclosed manifold to remain in fluid communication with the pressure transducer while in the contrast injection setting (FIGS. 4A-B) results in one less stopcock manipulation as compared to the conventional manifold.

### Stage #2

The second set of brackets outlines the valve manipulations required to fill the syringe with contrast. As shown in FIG. 3A, the presently disclosed contrast source valve (e.g., dual-check valve) positioned between the syringe and contrast source allows contrast to be drawn into the syringe barrel by retracting the plunger without any additional valve manipulations. By comparison, the conventional system in which the syringe is connected to the contrast source by a manifold, requires a valve to be rotated to an open position (“contrast open”) so that contrast can be drawn into the syringe (“fill’) and then rotated to a closed position (“contrast closed”) prior to injecting the contrast into the patient. Thus, two valve manipulations are required every time the syringe of the conventional fluid injection system is filled with contrast. As angiography procedures often require the syringe to be filled with contrast multiple times (N), the number of valve manipulations required by the conventional fluid injection system increases according to the formula (2×N). For example, an angiography procedure which requires the physician to load the syringe with contrast 25 times would represent a total of 50 additional valve manipulations.

### Stage #3

The third set of brackets outlines the stopcock manipulations required to inject contrast from the syringe into the patient's vasculature followed by a flush with saline solution.

Referring to the presently disclosed fluid injection system, contrast may be injected (“inject”) into the patient by simply depression the plunger of the syringe (FIG. 3B). The contrast injection step (“inject”) may be performed without any additional stopcock manipulation because the manifold is already in the contrast injection setting (FIGS. 4A-B), with the pressure transducer protected from high pressures associated with contrast injection by the integral pressure protection valve. It should be emphasized that the contrast injection setting provides the physician with the unique ability to repeatedly inject contrast in consecutive incremental amounts without having to continually switch the transducer between open (“transducer open”) and closed (“transducer closed”) configurations to monitor the patient's vascular pressure. If additional contrast is required, the physician simply retracts the plunger to re-fill the syringe with contrast. This level of operational flexibility allows the physician to administer the exact amount of contrast required to obtain, or maintain, a high resolution angiographic image. As recognized by those of skill in the art, this represents a savings in terms or patient prognosis, contrast usage and operating room expenses.

After the desired amount of contrast has been administered, the remaining stopcock manipulations are similar to those of Stage #1. That is, the stopcock is rotated 90° clockwise to place the manifold in the saline flush setting (FIGS. 5A-B), and saline is flushed (“flush forward”) through the catheter into the patient. The saline flush setting also provides the physician with the ability to repeatedly inject saline without having to continually switch the transducer between the open (“transducer open”) and closed (“transducer closed”) configurations to monitor the patient's vascular pressure. After the desired amount of saline has been administered, the stopcock is rotated 90° counter-clockwise to return the manifold to the contrast injection setting (FIGS. 4A-B) if subsequent injections of contrast are required. Again, the contrast injection setting allows the patient's vascular pressure to be monitored (“observe waveform”) without requiring any additional stopcock manipulations. Thus, two stopcock manipulations are required to inject contrast and saline into the vasculature of the patient using the presently disclosed manifold.

By comparison, the conventional fluid injection system requires the valve that protects pressure transducer to be rotated to a closed position (“transducer closed”) prior to injecting contrast (“inject”) into the patient. Unlike the presently disclosed fluid manifold, the physician cannot monitor the patient's vascular pressure between consecutive injections of contrast without rotating the valve that protects the pressure transducer between the open (“transducer open”) and closed (“transducer closed”) positions. After the desired amount of saline has been administered, the manifold is rotated to a position that establishes fluid communication with the saline source (“saline open”), and saline is flushed (“flush forward”) through the catheter into the patient. Again, unlike the presently disclosed fluid manifold, the physician cannot monitor the patient's vascular pressure between consecutive saline flushes without rotating the valve that protects the pressure transducer between the open (“transducer open”) and closed (“transducer closed”) positions. Following the saline flush, the manifold is rotated such that fluid communication with the saline source is blocked (“saline closed”). The valve that protects the pressure transducer is then rotated to an open position (“transducer open”) so that the patient's vascular pressure can be monitored (“observe waveform”). Thus, four stopcock manipulation are required to inject contrast and saline into the vasculature of the patient using the conventional manifold.

As angiography procedures often require multiple injections (M) of contrast and/or saline every time the syringe is filled, or refilled, with contrast, the number of additional stopcock manipulation required by the conventional manifold increases according to the formula (2×M). For example, an angiography procedure which requires the physician to perform 10 separate contrast injection (“inject”) and/or saline flush (“flush forward”) steps would represent a total of 20 additional stopcock manipulations. Naturally, this number will increase every time the syringe is re-filled with contrast (e.g., Stage #2).

When Stages 1-3 of Table 1 are considered together, the presently disclosed fluid injection system requires a minimum of five fewer valve and stopcock manipulations per angiography procedure, as indicated by the formula [Total Difference=1+4 Σᵢ^{N}Σⱼ^{M}Cᵢⱼ]. The number of valve manipulations increases every time the syringe is re-filled with contrast (N), and the number of stopcock manipulations increases every time contrast and/or saline are administered to the patient (M). Using the examples provided above, an angiography procedure that requires the syringe to be filled with contrast a total of 25 times (N), with 10 separate contrast injection (“inject”) and saline flush (“flush forward”) steps (M) per syringe, the conventional fluid injection system requires 1,001 additional valve and stopcock manipulations; e.g., [Total Difference=4(25×10)]. It should be emphasized that this number is merely exemplary, and that angiography procedures commonly require significantly higher numbers of syringe filling (N) and contrast/saline injecting (M) steps. As physician fatigue and the likelihood of mistakes increases with each valve and/or stopcock manipulation, the presently disclosed fluid injection system provides a dramatic reduction in manipulations that directly affects patient prognosis and operating costs.

TABLE 2

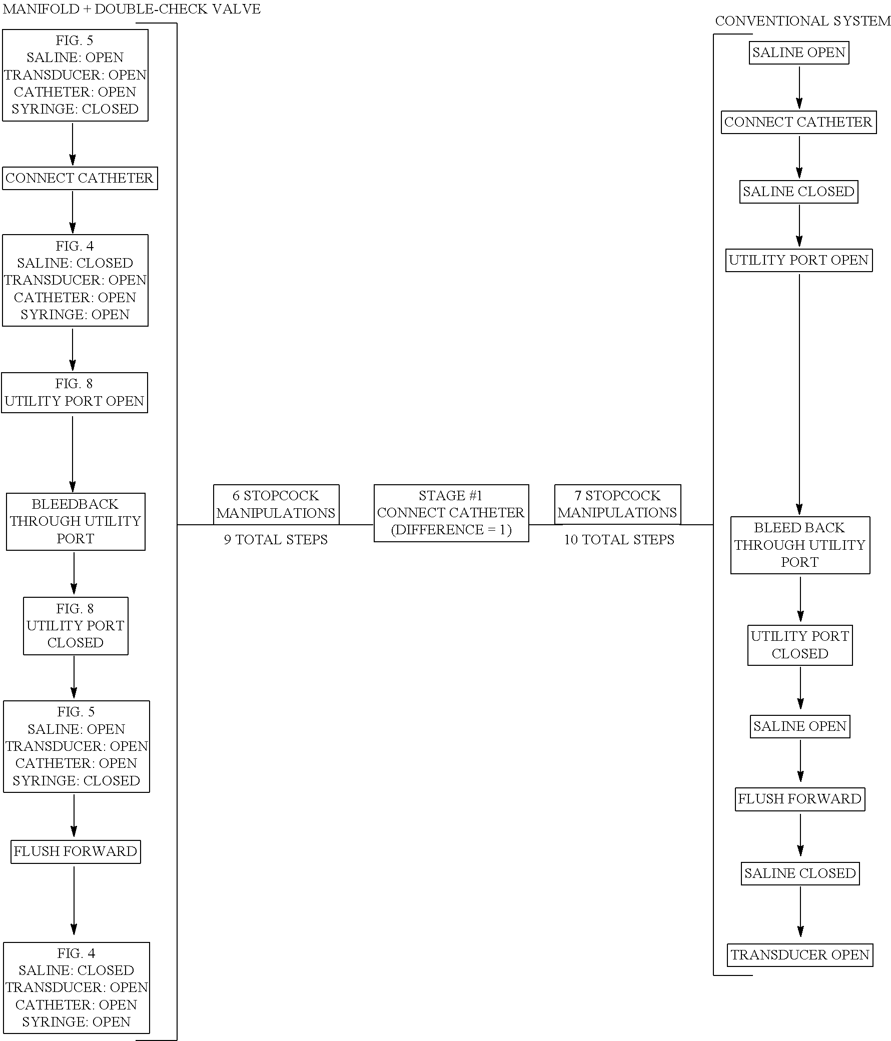

Table 2 outlines the stopcock manipulations required to connect a catheter to the manifold of a fluid injection system that further includes a utility port. As one might expect, the presence of a utility port increases the number of stopcock manipulations for the presently disclosed fluid injection system (6 stopcock manipulations) and the conventional fluid injection system (7 stopcock manipulations). Importantly, as demonstrated in Stage #1 of Table 1, the ability of the presently disclosed manifold to remain in fluid communication with the pressure transducer while in the contrast injection setting (FIGS. 4A-B) results in one less stopcock manipulation as compared to the conventional fluid injection system, regardless of the number of intervening stopcock manipulations.

All of the systems, assemblies and/or methods disclosed and claimed herein can be made and executed without undue experimentation in light of the present disclosure. While the present invention have been described in terms of preferred embodiments, it will be apparent to those of skill in the art that variations can be applied to the systems, assemblies and/or methods described herein without departing from the concept, spirit and scope of the invention. All such similar substitutes and modifications apparent to those skilled in the art are deemed to be within the spirit, scope and concept of the invention as defined by the appended claims.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A manifold, comprising:
a housing that defines intersecting first, second, third and fourth ports, wherein the non-intersecting end of each port is configured to be fluidly connected to an element that includes a fluid channel;
a rotatable stopcock comprising three fluidly connected conduits rotatably disposed at the intersection of the first, second, third and fourth ports; and
an actuator attached to the rotatable stopcock.

2. The manifold of claim 1, wherein the non-intersecting end of the first port is fluidly connected to a pressurized saline source.

3. The manifold of claim 1, wherein the non-intersecting end of the second port is fluidly connected to a dual-check valve.

4. The manifold of claim 1, wherein the non-intersecting end of the third port is fluidly connected to a pressure transducer.

5. The manifold of claim 1, wherein the non-intersecting end of the fourth port is fluidly connected to a catheter.

6. The manifold of claim 1, wherein the rotatable stopcock is configured to rotate about four stopcock positions within the manifold.

7. The manifold of claim 6, wherein the rotatable stopcock is rotated by turning the actuator attached to the rotatable stopcock.

8. The manifold of claim 6, wherein each of the four stopcock positions blocks fluid communication with a different one of the first, second, third and fourth ports.

9. The manifold of claim 8, wherein a first stopcock position places the second, third and fourth ports in fluid communication and blocks fluid communication with the first port.

10. The manifold of claim 8, wherein a second stopcock position places the first, third and fourth ports in fluid communication and blocks fluid communication with the second port.

11. The manifold of claim 8, wherein a third stopcock position places the first, second and fourth ports in fluid communication and blocks fluid communication with the third port.

12. The manifold of claim 8, wherein a fourth stopcock position places the first, second and third ports in fluid communication and blocks fluid communication with the fourth port.

13. A fluid injection system, comprising:
a syringe assembly;
a contrast source valve fluidly connected to the syringe assembly;
a manifold fluidly connected to the contrast source valve; and
a catheter fluidly connected to the manifold.

14. The fluid injection system of claim 13, wherein the contrast source valve is a dual-check valve.

15. The fluid injection system of claim 13, wherein the contrast source valve is fluidly connected a contrast source.

16. The fluid injection system of claim 13, wherein the manifold comprises:
a housing that defines intersecting first, second, third and fourth ports, wherein the non-intersecting end of each port is configured to be fluidly connected to an element that includes a fluid channel;
a rotatable stopcock comprising three fluidly connected conduits rotatably disposed at the intersection of the first, second, third and fourth ports; and
an actuator attached to the rotatable stopcock.

17. The fluid injection system of claim 16, wherein the non-intersecting end of the first port is fluidly connected to a pressurized saline source.

18. The fluid injection system of claim 16, wherein the non-intersecting end of the second port is fluidly connected to the syringe assembly.

19. The fluid injection system of claim 16, wherein the non-intersecting end of the third port is fluidly connected to a pressure transducer.

20. The fluid injection system of claim 16, wherein the non-intersecting end of the fourth port is fluidly connected to the catheter.

21. The fluid injection system of claim 13, further comprising a utility port fluidly connected between the contrast source valve and the manifold.
